(19) United States
(12) Patent Application Publication
(10) Pub. No.: US 2008/0281249 A1
(43) Pub. Date: Nov. 13, 2008

(54) Valves and Conduits for Vascular Access

(76) Inventor: Michael Gertner, Menlo Park, CA (US)

Correspondence Address: MICHAEL GERTNER, P.O. BOX P, MENLO PARK, CA, 94026, US

(21) Appl. No.: 11/930,064

(22) Filed: Oct. 30, 2007

Related U.S. Application Data
(60) Provisional application No. 60/863,559, filed on Oct. 30, 2006.

(51) Int. Cl. A61M 1/14

(52) U.S. Cl. 604/6.16

(57) ABSTRACT

A dialysis fistula graft comprising a valve mechanism with a valve member which is substantially flush with blood flowing in a blood vessel in the closed configuration and allows for blood flow into the fistula graft when the valve member is in the open position. In one embodiment, a subcutaneous access to the valve is provided in which the valve can be opened or closed by applying force through the skin of a patient.

### PRIORITY DATA

This application claims priority to U.S. provisional patent application Ser. No. 60/863,559.

### INCORPORATION BY REFERENCE

This provisional patent application incorporates the following patents and applications by reference:
Ser. No. 11/425,106
Ser. No. 10/177,721
Ser. No. 10/456,697

### BACKGROUND OF THE INVENTION

1. Field of the Invention

This invention pertains generally to hemodialysis, and more particularly to hemodialysis systems and methods including A-V fistula grafts, A-V fistula graft treatment systems, systems for treating a condition associated with an AV-fistula graft, and an A-V fistula graft systems.

2. Description of the Background Art

Renal disease and deficiency has long been a significant problem that continues to plague an enormous population of patients, and the related cost of treatment continues as an ever growing burden on modern society as a whole. For example, in 1996, there were 250,000 patients in the US with end stage renal disease (ESRD), a number expected to grow by 10-15% per year over the next 20 years primarily as a result of an aging population and advances in treatments for other diseases. The cost of ESRD in the US was $20 billion in the year 2000, 5% of all Medicare resources.

Dialysis involves cannulation of the vascular system for extracorporeal flow of blood through a dialysis machine, which acts as a filter. To filter the blood efficiently, the dialysis machine requires 300-400 ml/min of flow for approximately three hours three times per week. To supply this high a flow rate, a large vein is required which will provide a flow rate of at least 300-400 ml/min. Otherwise, the vessel will collapse as the dialysis machine pulls out blood.

Various central venous devices and methods have been disclosed that provide this generally required level of flow. Examples of such devices include, without limitation, “TESSIO™” and “QUINTON™” catheters, which are commercially available from Medical Components and Kendall (owned by Tyco International) corporations, respectively. In general, these devices are inserted into the subclavian or internal jugular veins, communicate exteriorly of the patient, and at best are considered “semi-permanent” devices in that their longevity is limited, generally lasting up to a typical maximum of about 4 months.

The primary “long-term” solution generally involves gaining peripheral vessel access, most typically in an accessible region of a patient's arm. This generally requires a surgical procedure, wherein an artery is surgically attached to a vein, either directly or via an artificial conduit that creates an arterio-venous fistula, such as for example a conduit made from polytetrafluoroethylene (PTFE) or a woven polyester such as Dacron™. This procedure essentially short-circuits the normal blood path to the hand, and can provide a flow of approximately 1 liter/min. The conduit fistula is typically coupled to the corresponding arteries, such as by suturing, at locations called arterial and venous (respectively) “anastomoses”. According to the typical dialysis procedure, the dialysis fistula is connected to a dialysis machine via dialysis needles that puncture the fistula conduit at a location between the anastomoses. Blood traveling from the fistula through the needles are carried by tubing into a dialyzer, which cleanses the blood by removing waste matter, and returns the blood via another needle to the fistula. A typical blood cleansing procedure lasts about 3 hours, after which the dialysis needles are removed and pressure is held at the site of needle entry. Most patients require dialysis about three times per week. A damaging process called “intimal hyperplasia” often begins at the time of surgery, and continues undisturbed in most cases until it leads ultimately to failure of the access fistula.

In common practice, an artificial conduit is used 70% of time, as has been previously disclosed by Stehman-Breen et al., “Determinants of type and timing of initial permanent hemodialysis vascular access,” Kidney International, 57(2000) 639-645. However, about 50% of these grafts have been observed to malfunction within 2 years of implantation, as has been previously published by Diskin, C J et al., “Pharmacologic Intervention to Prevent hemodialysis Access Thrombosis,” Nephron 1993:64(1-26). A study by Tellis, V. A. et al., “Expanded Polytetrafluoroethylene Graft Fistula for Chronic Hemodialysis,” Ann. Surg., Vol 189(1), 1979, pp 101-105, revealed a 62% primary patency rate in PTFE grafts. It is not believed that this number has changed significantly since this study despite enormous advances in technology in other fields. The disclosures of the reference articles provided in this paragraph are incorporated herein in their entirety by reference thereto.

The creation of such a fistula increases flow to the arm and hence to the dialysis machine. The major problem in permanent dialysis access is the longevity of the fistula. With current methods, fistula survival is generally about 8-12 months with artificial conduits, and generally about 2-3 yrs with autogenous conduits. In fact, it is believed that about 3 “revision procedures” are required for every new fistula created. Each revision procedure requires a new access site on the patient's body. While the new fistula matures, a semi-permanent catheter needs to be placed in a large central vein. This usually leads to substantial morbidity, cost, and physician frustration; and in 1993 vascular access was described as a $1 billion problem. In a study published by Arora, P. et al., “Hospital Utilization among Chronic Dialysis Patients,” J. Am. Soc. Nephrol., 11: 740-746, 2000, 36% of all hospital admission for dialysis patients was for matters related to access. Patients on dialysis require an average of about 2.2 hospitalizations and about 14.8 hospital days per year related to dialysis access. Many patients die secondary to lack of access. In an earlier study cited by Swapna, J. et al. in “Vascular Access Problems in Dialysis Patients,” Heart Disease 2001; 3:242-247, about 18% of deaths in the dialysis population was due to lack of access. Though this number may have decreased in recent years as devices and techniques improve, it still remains a significant issue that deserves attention. The disclosures in the reference articles cited in this paragraph are herein incorporated in their entirety by reference thereto.

Morbidity related to fistulas fall into several categories, the most common of which (e.g. about 95%) is clotting of the graft. Infection occurs in 18% of complications and pseudoaneurysm in about 2%. The clotting pathophysiology can be further subdivided into clotting secondary to a venous stenosis (about 55% of cases), or secondary to an arterial stenosis (about 10% of cases). Other reasons for clotting include hypotension and pressure to curtail bleeding following a dialysis session.

Access to a fistula currently entails placement of a needle through the skin and into the fistula with subsequent attachment to a dialysis machine. The placement of the needle is not standardized with respect to the fistula, being placed in a different spot in the graft each time, resulting in disruption of the ultrastructure of the material over time. Twenty (20%) percent of fistula failures occur at the site of needle entry and manifest as thrombosis, pseudoaneurysms, and aneurysms. Furthermore, at least about 10 minutes of pressure is usually required to prevent hematoma formation at the access site, which may itself lead to a thrombosis.

Various devices and methods intended to treat AV-fistula stenoses with localized energy delivery have been disclosed. For example, several devices and methods have been disclosed for delivering ultrasound energy to an anastomosis region.

At least one example of this type is intended to deliver ultrasound energy to the area of an existing fistula thrombosis in combination with delivery of an echo contrast agent into the area to enhance the ultrasonic affects at the thrombosis. The ultrasound energy may be delivered transcutaneously to the area, or intravascularly such as by use of a miniature ultrasonic transducer located on a catheter inserted within the fistula. However, this particular technique suffers by the rapid clearance that the contrast agent may experience from the area in a blood flow environment. Also, this example does not provide for a device or method for using energy delivery for regular preventative maintenance of fistula patency, such as to prevent thrombus formation or adhesion in the fistula, or to prevent or treat neo-intimal hyperplasia.

At least one other example also includes a system and method for delivering ultrasound to the anastomotic junctions of fistulas in order to inhibit substantial neo-intimal hyperplasia by use of an ultrasound transducer located on an internal catheter probe within the fistula, or with a focused ultrasound transducer assembly associated with an external ultrasound energy source. However, this example does not provide for prevention or removal of thrombus. In addition, the internal catheter aspect of this example requires an active ultrasound energy source to be located on the catheter itself, which results in significant complexity and cost that may be prohibitive to regular maintenance use as a disposable assembly. The active source in addition may limit the ability to make such a catheter sufficiently small to be inserted into a fistula lumen through certain needles such as certain hemodialysis needles.

In addition to the limitations of the previous ultrasound energy delivery examples just described, they also do not provide for a system or method for actuating an treatment device within a fistula to deliver vibratory or other energy to tissues by exposing the treatment assembly to an applied energy field from a remotely located energy source outside of the fistula, such as externally of the patient and transcutaneously across a skin barrier. Nor do these previous techniques provide for the ability to deliver an energy delivery treatment assembly into a fistula through a hemodialysis needle such that additional punctures of the fistula are not required. Still further, these previous techniques also do not provide for an energy delivery treatment assembly secured to and implanted with a fistula graft. Nor do these techniques provide for other forms of energy delivery than ultrasound into problematic areas associated with fistula grafts in order to provide therapy to a patient.

Another example of a previously disclosed device system and method provides for delivery of a doppler ultrasound monitoring transducer into a fistula through a hemodialysis needle. However, the doppler device and method of this example does not deliver energy into the fistula in order to affect treatment or prevention of stenosis associated with the fistula. Other beneficial forms of energy delivery other than doppler ultrasound also are not provided according to this example. Moreover, there is no provision for applying an energy field from outside of a fistula to actuate energy delivery from a treatment assembly located within the fistula.

Various previous disclosures that provide additional background information and further illustrate the context of various aspects of medical device systems and methods herein summarized or described include the following issued U.S. patents: U.S. Pat. No. 3,225,129 to Taylor et al.; U.S. Pat. No. 3,953,566 to Gore; U.S. Pat. No. 3,962,153 to Gore; U.S. Pat. No. 4,187,390 to Gore; U.S. Pat. No. 4,267, 863 to Burelle; U.S. Pat. No. 4,536,018 to Patarcity; U.S. Pat. No. 4,787,921 to Shibata et al.; U.S. Pat. No. 6,019,788 to Butters et al; U.S. Pat. No. 6,102,884 to Squitieri; and U.S. Pat. No. 6,153,252 to Hossainy et al. The disclosures of these references are herein incorporated in their entirety by reference thereto.

Other previously disclosed devices and methods that disclose additional background information related to at least one of fistulas, valves, renal interventions, or dialysis may be reviewed by reference to the following issued U.S. patents: U.S. Pat. No. 4,822,341 to Colone; U.S. Pat. No. 5,454,374 to Omachi; U.S. Pat. No. 5,562,617 to Finch et al.; U.S. Pat. No. 5,690,115 to Feldman et al.; U.S. Pat. No. 5,702,715 to Nikolaychik et al.; U.S. Pat. No. 5,879,320 to Cazenave; U.S. Pat. No. 6,086,573 to Siegel et al.; U.S. Pat. No. 6,113,570 to Siegel et al.; U.S. Pat. No. 6,177,049 to Schnell et al. U.S. Pat. No. 6,319,465 to Schnell et al.; and U.S. Pat. No. 6,387,116 to McKenzie et al. The disclosures of these references are also herein incorporated in their entirety by reference thereto.

Despite certain advances that may have been provided by various of the disclosures cited above, there are still many needs that have not yet been adequately met.

There is still a need for a hemodialysis system and method that provides for improved longevity and patency of AV-fistula implants.

There is in particular still a need for a hemodialysis system and method that substantially prevents or removes occlusive stenoses associated with AV-fistula implants.

There is also a need to accomplish the foregoing while minimizing morbidity and without the use of substantially invasive interventions.

### SUMMARY OF INVENTION

In one embodiment, a valve for placement at the anastomosis between a vascular graft and a blood vessel is described in which the closed position of the valve is characterized by a valve member which is flush with blood flow inside the blood vessel so that minimal disturbance of the flow occurs when the valve is in the closed position. In the open position, the valve allows for flow out of the artery and into the vascular graft.

### DESCRIPTION OF INVENTION

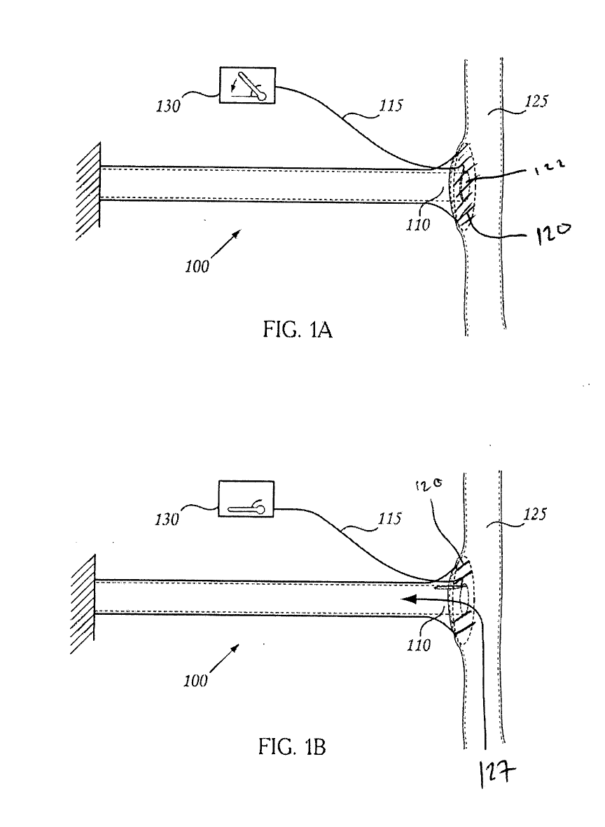

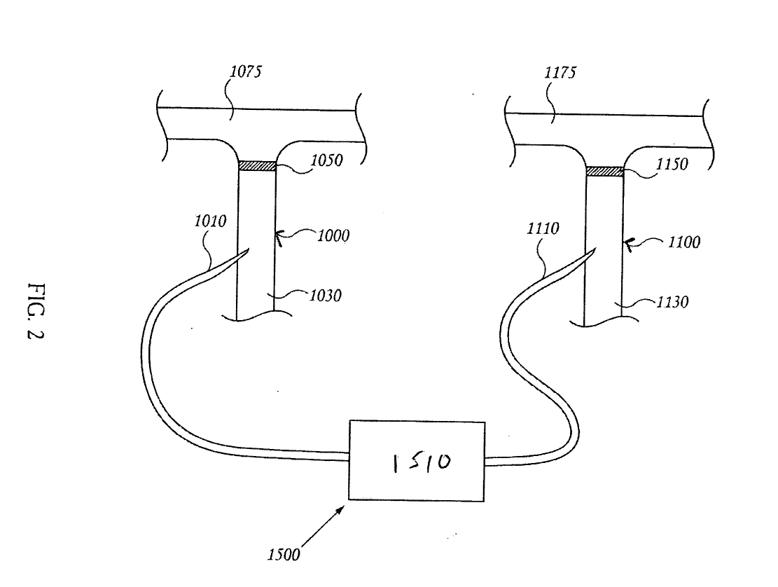

In one embodiment of this invention, a valve mechanism 110 applied to a vascular graft 100 (FIG. 1a-b). This valve 110 is attached to an arm 115 which enables opening and closing of the valve 110 through application of force to the arm 115 using an actuation mechanism 130; the force applied through the arm 115 is translated to the valve mechanism 110.

Valve 110 is flush with the wall of the vessel in one embodiment; ideally, valve 110 is similar to a trap door in a room in which the trap door is flush with the wall of the room until the door 122 is opened via a hinge. The force translated by the arm to the door which creates a suction force on the door and thence on film 120 formed over the valve when the graft is not in use. The force applied to the valve 110 to open the valve breaks the film 120 over the valve 120 so that flow 127 is now achievable through the valve 110 and graft 100.

In another embodiment, the valve 110 contains an automated force or energy mechanism; in one example, a piezoelectric, or other vibratory element, which can be activated through the actuating mechanism 130 to break open the film 120 allowing the valve 110 to open and thence blood to flow 135 into and through the graft.

FIG. 2 depicts a novel dialysis system wherein the arterial inflow has a graft 1000 and the venous end has a separate graft 1100; these are independent access sites, each with its own graft and each with its own anastamosis with valve (or half graft). For example, the artery can be a radial artery on one arm and the vein can be a vein on the other arm. One graft 1000 is attached to one vessel and contains its own valve 1050, 1150 and opening mechanism similar to those shown in FIG. 1 (in FIG. 1, the graft has a valve at both ends). With the configuration in FIG. 2, artery 1075 and vein 1175 can be located at disparate regions of the patient, making more access sites possible.

FIG. 2 depicts a system 1500 for hemodialysis in which grafts 1000 and 1100, representing an arterial and venous access respectively, are each placed in different regions for a given patient. For example, the arterial side 1000 can be placed in one arm and the venous side in another arm or a leg. When accessed with different dialysis needles 1010 and 1110, a shunt is created as if the access was continuous. Blood from the patient is pumped through a dialyzer 1510 and then back into the patient. Similar to the proposed methodology in FIG. 1, each access (now independent) site 1000, 1100 is filled with an anticoagulant solution (or other solution) after access and hemodialysis; the doors 1050, 1150 are also closed. A difference between the hemodialysis access devices in FIGS. 1 and 2 is that the devices 1000, 1100 in FIG. 2 have blind ends 1030, 1130 which are essentially fluid filled reservoirs.

A method of creating a dialysis access in a patient includes creating arterial and/or venous valves with doors substantially flush with the vessel and elongated tubular components extending from the valves and wherein each valve is placed in a different body region and there is not a continuous vascular conduit.

### Blood Vessel Valves

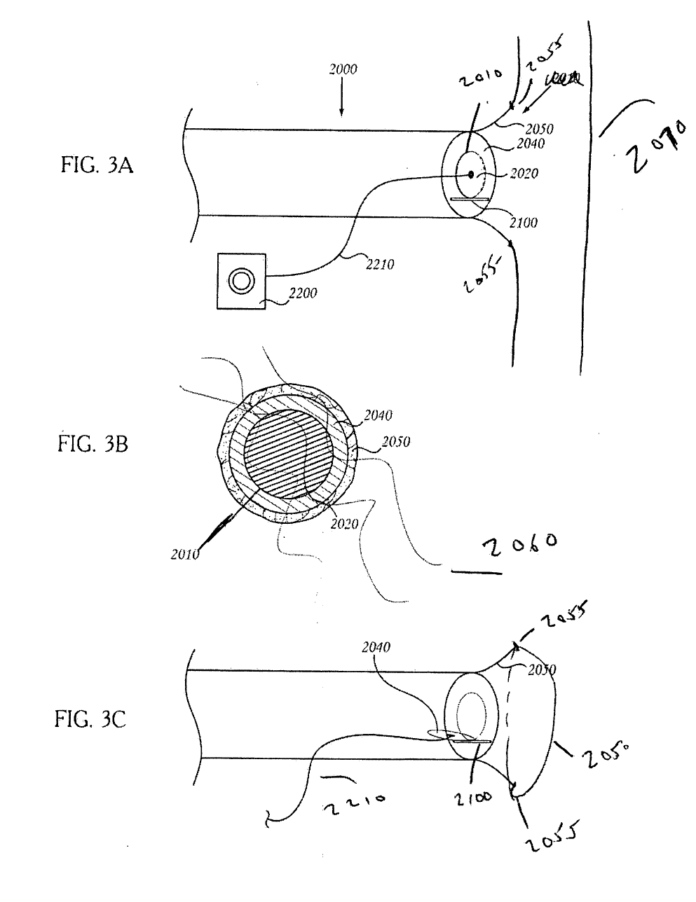

FIGS. 3a-c depict a type of valve assembly 2010. The valve 2010 sits at the end of the graft 2000. The components of the valve assembly 2010 include a seating for the valve 2040 and the valve door 2020. The tolerance between the valve door 2020 and the seating of the valve 2040 can be tens of microns up to the millimeter range. At the millimeter range of tolerance, the fluid from inside the graft 2000 mixes with the blood and other components at the valve-blood 2010 interface. For example, if the solution inside the graft is an anticoagulant solution, then the anticoagulant solution can leak into the region around the valve assembly to create a desired biologic response such as prevention of biologic films or clots. Region 2055 represents the region where suturing of the valve occurs on the artery 2010. In this embodiment, the valve 2040 is attached to the graft 2000 independently of the surgical procedure.

Material 2050 is part of the valve assembly 2010 and forms what is known in the art as a Carrell Patch, now a valved Carrell Patch. The patch surrounds the valve and is useful because it takes variability out of the valve placement. Without the surrounding Carell patch, the valve itself would have to been sewn to the artery whereas with the carell patch, the valve is attached prior to vessel anastomosis and tested; then it is sewn to the artery 2070.

The valve door 2020 and/or seating 2040 can be manufactured from materials such as Teflon or other biocompatible biologic polymers. Door 2020 and/or seating 2040 can also be manufactured from stainless steels, titanium, and cobalt chromium. Door 2020 and/or seating 2040 can be manufactured with patterns to induce or inhibit biologic growth.

Attached to the edge of the seating 2040 is a rim of graft material 2050 similar to that of the tubular portion of the graft 2000. Examples of this material include PTFE, Dacron, Polyurethane, or combinations of these materials. This material and valve assembly 2050 forms the interface with the blood vessel to which valve assembly 2010 is attached. Material 2050 allows for pre-assembly and attachment of the valve assembly 2010 and also facilitates attachment to the blood vessel during surgery.

Hinge 2100 allows the door 2020 to move from the closed position, in which it is parallel to blood flow in the blood vessel, to an open position (FIG. 3C) through which blood 127 travels. Actuation device 2200 is accessible through the skin of the patient without penetrating the skin of the patient. Cable 2210 is the connector between actuation device 2200 and the valve assembly 2010.

### Actuation Mechanism

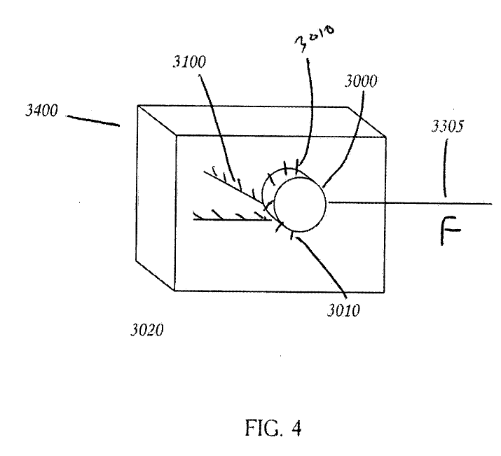

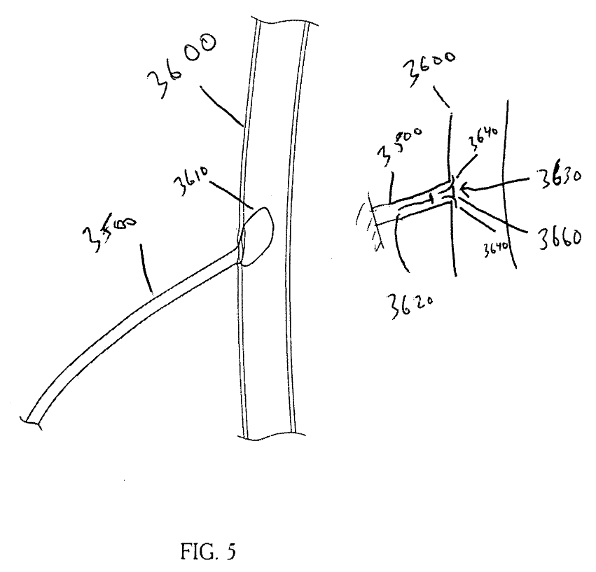

FIG. 4 depicts an embodiment of a subcutaneous actuating mechanism 3400 for the valve shown in previous figures. Teeth 3010 enable the rotary wheel 3000 to move incrementally as force F is applied to the levers 3100. The cable (3305) transfers the force F from the gears to the valve door. The actuating mechanism 3400 is covered with a biologically compatible flexible polymer such as silicone and can be accessed through the skin to open the valve (2200 in FIG. 3). For example, mechanism 3400 can be actuated by pinching the skin and squeezing the levers to engage the teeth and open or close the valve. Alternatively, a needle can be inserted through the skin to actuate the valve opening mechanism 3400. Mechanism 3400 can optionally have a safety lock in it so that it cannot be accessed accidentally. The lock can be accessed or turned on by the physician or medical technician only at the time of dialysis. A radiofrequency type security system can be used to engage and enable the ability to actuate the lever system 3100.

### Percutaneous Delivery

In one embodiment, a percutaneous delivery technique is described. In this embodiment, a catheter 3500 is inserted through the skin and into a blood vessel 3600. The distal end of the catheter contains a valve to be deployed inside the blood vessel 3600. A plunger 3620 from within the catheter 3500 can be used to deploy the valve 3630. Valve 3630 contains a door 3660 with flanges 3640 to grip the walls of the vessel after deployment. A percutaneous deployment technique can be used for existing dialysis grafts so as to retrofit them for on-off abilities.

### Valve With Seating Fixture

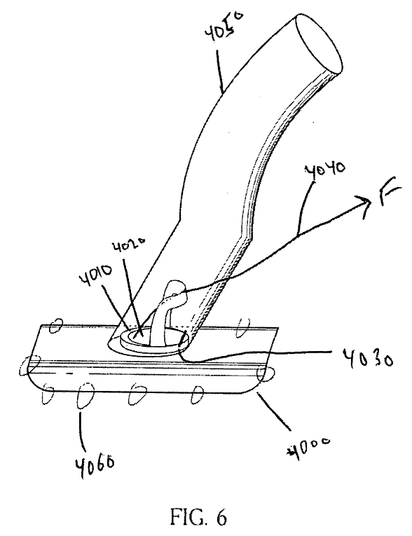

FIG. 6 depicts a carell patch 4000 with attached valve 4010 within a vascular graft 4050. Attached valve 4010 contains a door, a trap door 4020, and a hinge joint 4030 to enable opening of the door with cable 4040. Door 4020 is flush with patch 4000. Patch 4000 is sewn to the blood vessel via sutures 4060. An advantageous feature of this embodiment is that the valve 4010 can be attached to the graft 4050 prior to delivery to the surgeon. It can also be sterilized prior to delivery as well. The surgeon then sews the Carell patch portion of the valve to the vessel in a similar manner to other procedures. Cable 4040 can be attached to a subcutaneous mechanism to open the valve using force F.

### Valve With Angled Connector

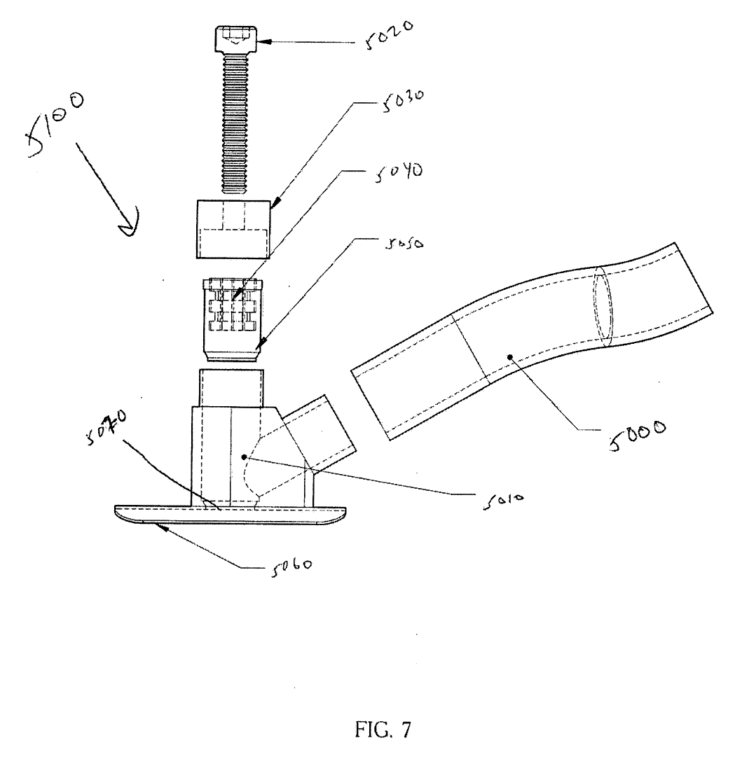

FIG. 7 depicts another embodiment of a valve configuration 5100 with a carell patch 5060 attached. An angled connector 5010 is included in the valve assembly 5100. The distal end 5070 of the angled connector 5010 is adapted to receive plunger 5040. When plunger 5040 is received by the angled connector 5010, the plunger 5040 can be flush with the carell patch 5060 or it can extend beyond the Carell patch 5060. The material of the plunger 5040 can be silicone, polyurethane, PEEK, PTFE, or combinations of polymers. The plunger 5040 or the Carell patch can be made from a material to inhibit endothelial growth. The plunger material can be hydrophobic or hydrophilic or can have a molecule bonded to its surface.

1. An accessory for a hemodialysis access graft comprising:
A patch of hemocompatible material with a hole for blood flow into the hemodialysis access graft;
A valve mechanism comprising an open configuration and a closed configuration wherein the closed configuration is configured to deliver a valve member which substantially covers said hole;
and wherein, in the open position, the valve member pulls away from the patch such that blood can flow through the hole.

2. The system of claim 1 wherein said valve member is substantially flush with said patch when in the closed configuration.

3. The system of claim 1 wherein said valve member extends beyond said patch when in the closed position.

4. The system of claim 1 wherein said valve member comprises a material which inhibits endothelial growth.

5. A method of hemodialysis comprising: implanting the patch of claim 1 on an artery and the patch of claim 1 on a vein wherein a conduit does not connect the artery and the vein; performing hemodialysis treatment on a patient wherein during the hemodialysis treatment, all blood flows from the artery, into a dialyzng machine and then into the vein without direct shunting between the artery and the vein.

6. A hemodialysis system comprising: a hemodialysis access graft comprising a valve at either or both the arterial or venous anastomosis; a connection between the valve and a subcutaneous actuator wherein the subcutaneous actuator is actuated through force applied through the skin and to the actuator to initiate actuation of the valves.

7. The system of claim 5 wherein said subcutaneous actuator comprises a lever and gear teeth and wherein the valve is progressively opened by force applied to the lever and gear through the skin.
